(19) United States
(12) Patent Application Publication
(10) Pub. No.: US 2018/0147129 A1
(43) Pub. Date: May 31, 2018

(54) MILD COSMETIC CLEANING COMPOSITION WITH GOOD FOAM PROPERTIES

(71) Applicant: Henkel AG & Co. KGaA, Duesseldorf (DE)

(72) Inventors: Heike Schelges, Willich (DE); Laurine Figuet, Saulchery (FR)

(73) Assignee: Henkel AG & Co. KGaA, Duesseldorf (DE)

(21) Appl. No.: 15/574,842

(22) PCT Filed: Apr. 19, 2016
(86) PCT No.: PCT/EP2016/058624

§ 371 Date: Nov. 17, 2017

(30) Foreign Application Priority Data
May 20, 2015 (DE) 10 2015 209 172.4

(51) Int. Cl. A61K 8/46; A61K 8/42; A61K 8/86; A61Q 19/10

(52) U.S. Cl. CPC A61K 8/463; A61K 8/42; A61K 2800/48; A61Q 19/10; A61K 8/86

(57) ABSTRACT

The present disclosure relates to a cosmetic cleansing composition which contains at least one nonionic surfactant having the following formula (I)

R¹CO—N(R²)—CH₂—CH(OH)—CH(OH)—CH(OH)—CH(OH)—CH₂OH (I),

wherein R¹CO denotes a straight-chained or branched, saturated or unsaturated C₈-C₂₂-acyl radical and R² denotes a C₁-C₄-alkyl radical and at least one polyethylene glycol of a molar mass M_{W} with an average mass of 1,000,000 to 10,000,000 Dalton is contained. The cosmetic cleansing compositions are storage- and temperature-stable and generate large amounts of a dense, creamy foam which can be distributed well and glides on the skin and can also be rinsed off well and leaves a smooth, soft and nourished skin feeling. The cleansing agents also have a very pleasant texture. With an additional content of special thickening agents, the rheology and removability of the cleansing agents from the respective container can be controlled and/or adjusted particularly well.

### CROSS-REFERENCE TO RELATED APPLICATION

This application is a U.S. National-Stage entry under 35 U.S.C. § 371 based on International Application no. PCT/EP2016/058624, filed Apr. 19, 2016, which was published under PCT Article 21(2) and which claims priority to German Application no. 10 2015 209 172.4, filed May 20, 2015, which are all hereby incorporated in their entirety by reference.

### TECHNICAL FIELD

The present disclosure is within the field of cosmetics and pertains to cleansing compositions, which contain polyethylene glycol of a high molar mass in addition to anionic surfactants and special nonionic surfactants.

### BACKGROUND

Cosmetic cleansing agents usually contain surfactants. Due to their outstanding foam properties, anionic surfactants are mostly used as primary surfactants, which, however, in higher quantities can have the disadvantage that they macerate the skin and remove lipids from the outer skin layers. Consequently, the (sensitive) skin can become dry, chapped and cracked after frequent cleansing with commercially available cleansing agents, unless lipids are re-introduced in the form of creams, lotions and/or nourishing shower baths after or during the cleansing.

In the past, numerous attempts were made to produce milder cosmetic cleansing agents which dry out the skin less and render an additional treatment step with care products unnecessary.

For this purpose, high amounts of anionic surfactants were normally omitted in favor of higher shares of milder nonionic and-or amphoteric surfactants, which often resulted in products with unsatisfactory foam properties.

Low-surfactant, mild cleansing compositions received poor evaluations by consumers with regard to the amount of formed foam and in regard to the haptic properties and creaminess of the resulting foam.

In DE 102006030091A1, polyethylene glycols were proposed for improvement of foam properties, particularly for improvement of the creaminess/richness of the foam of cosmetic cleansing compositions.

However, these products do not always have a satisfactory skin feeling during and after use. The products also needed improvement in regard to the so-called “high slip” skin feeling (strongly pronounced smoothing of the skin already during use), in particular.

Products with “high slip” are particularly desirable, because they are associated with special care by consumers.

Therefore, there is still a requirement for mild cosmetic cleansing agents with very good foam properties which simultaneously improve the skin feeling during and/or after use of the agent.

The task of the present disclosure was to a provide mild, foamy cosmetic cleansing agent which forms a foam with excellent haptic properties and can be distributed well over the skin without leaving behind a slippery, dry or tight feeling.

The cleansing agents should have extreme storage stability and easy to produce in an energy-saving production process and adjustable with regard to the desired viscosity.

For adjustment of the viscosity, the products should not develop any undesired rheological properties in the desired viscosity range. In particular, the formation of threads (good tearing of the product is desirable) or poor distributability due to gelling of the product with the addition of water should be avoided.

### BRIEF SUMMARY

Cosmetic cleansing compositions are provided herein. In an embodiment, a cosmetic cleaning composition includes (a) at least one anionic surfactant, (b) at least one nonionic surfactant, and (c) at least one polyethylene glycol of a molar mass M_{W} with an average mass of from about 1,000,000 to about 10,000,000 Dalton. The at least one nonionic surfactant (b) has the following formula (I)

R¹CO—N(R²)—CH₂—CH(OH)—CH(OH)—CH(OH)—CH(OH)—CH₂OH (I),

where R¹CO denotes a straight-chained or branched, saturated or unsaturated C₈-C₂₂-acyl radical and R² denotes a C₁-C₄-alkyl radical.

In another embodiment, a cosmetic cleansing composition includes (a) at least one anionic surfactant, (b) at least one nonionic surfactant, (c) at least one polyethylene glycol of a molar mass M_{W} with an average mass of from about 1,000,000 to about 10,000,000 Dalton, (d) from about 0.10 to about 5.00 wt. % of at least one associative thickening agent, and (e) optionally, from about 0.10 to about 5.00 wt. % of at least one fatty alcohol polyglycol ester. The at least one anionic surfactant (a) is selected from alkyl(ether)sulfate surfactants, sulfosuccinate surfactants and/or isethionate surfactants. The at least one nonionic surfactant (b) has the following formula (I)

R¹CO—N(R²)—CH₂—CH(OH)—CH(OH)—CH(OH)—CH(OH)—CH₂OH (I),

where R¹CO denotes a straight-chained or branched, saturated or unsaturated C₈-C₁₈-acyl radical and R² denotes a methyl radical. The optional (e) at least one fatty alcohol polyglycol ester has the general formula (IV),

R—O—(CH₂—CH₂—O)ₙ—H (IV)

wherein R denotes a straight-chained or branched, saturated or unsaturated C₈-C₂₄-alkyl radical and n denotes a number from 1 to 10.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

It has now been found, surprisingly, that the aforementioned tasks are solved in an outstanding manner with the aid of cosmetic cleansing agents which contain a combination of mild surfactants selected from anionic surfactants and special nonionic surfactants, and high molecular weight polyethylene glycols.

The cleansing agents are storage- and temperature-stable and generate large amounts of a dense, creamy foam which can be distributed well and glides on the skin and can also be rinsed off well and leaves an especially smooth (high slip), soft and nourished skin feeling.

The cleansing agents also have a very pleasant texture. With an additional content of special thickening agents, the rheology and removability of the cleansing agents from the respective container can be controlled and/or adjusted particularly well.

Therefore, a first subject of the present disclosure is a cosmetic cleansing composition containing

a) at least one anionic surfactant,
b) at least one nonionic surfactant having the following formula (I)

R¹CO—N(R²)—CH₂—CH(OH)—CH(OH)—CH(OH)—CH(OH)—CH₂OH (I),

where

R¹CO denotes a straight-chained or branched, saturated or unsaturated C₈-C₂₂-acyl radical and

R² denotes a C₁-C₄-alkyl radical and

c) at least on polyethylene glycol of a molar mass M_{W} with an average mass of from about 1,000,000 to about 10,000,000 Dalton.

The cosmetic agents as contemplated herein contain the active ingredients a) to c), preferably in a cosmetic carrier, preferably in an aqueous carrier.

In the context of the present disclosure, an aqueous carrier is preferably understood to mean an aqueous or aqueous-alcoholic carrier. Preference is given to an aqueous carrier containing at least about 60 wt. %, preferably at least about 65 wt. %, more preferably at least about 70 wt. %, particularly at least about 75 wt. % water.

Furthermore, the cosmetic carrier can contain from about 0.01 to about 40 wt. %, preferably from about 0.05 to about 30 wt. % and more preferably from about 0.1 to about 20 wt. % of at least one alcohol. Suitable alcohols include ethanol, ethyldiglycol, 1-propanol, 2-propanol, isopropanol, 1,2-propylene glycol, 1,3-propylene glycol, 1-butanol, 2-butanol, 1,2-butanediol, 1,3-butanediol, 1,4-butanediol, 1-pentanol, 2-pentanol, 1,2-pentanediol, 1,5-pentanediol, 1, hexanol, 2-hexanol, 1,2-hexanediol, 1,6-hexanediol, glycerol, polyethylene glycol, sorbitol, sorbitan, benzyl alcohol, phenoxyethanol or mixtures of said alcohols. Particular preference is given to the aqueous alcohols. Particular preference is given to ethanol, 1,2-propylene glycol, glycerol, benzyl alcohol and/or phenoxy ethanol, as well as mixtures of these alcohols.

An optimal balance between mild and high amounts of foam of the cosmetic cleansers as contemplated herein could be achieved to a particular extend with careful selection of the surfactant amounts, the surfactant types and the weight ratios of the at least one anionic surfactant a) to achieve the at least one nonionic surfactant b) according to formula (I).

Therefore, a first particularly preferred embodiment of the present disclosure is exemplified in that the cosmetic cleansing compositions contain (relative to their total weight)

from about 1.00 to about 15.00 wt. %, preferably from about 3.00 to about 12.50 wt. % and particularly from about 5.00 to about 10.00 wt. % of at least one anionic surfactant a),

from about 0.50 to about 7.50 wt. %, preferably from about 0.75 to about 6.00 wt. % and particularly from about 1.00 to about 5.00 wt. % of at least one nonionic surfactant (b) of the aforementioned formula (I), and

from about 0.01 to about 1.00 wt. %, preferably from about 0.03 to about 0.50 wt. % and particularly from about 0.05 to about 0.30 wt. %, of at least one polyethylene glycol of a molar mass M_{W} with an average mass of from about 1,000,000 to about 10,000,000 Dalton.

The suitable anionic surfactants a) which can be used in the cleansing compositions as contemplated herein include, for example:

linear and branched fatty acids having from 8 to about 30 carbon atoms (soaps),

ether carboxylic acids having the formula R—O—(CH₂—CH₂O)ₓ—CH₂—COOH, wherein R is a linear or branched, saturated or unsaturated alkyl group having from 8 to about 30 carbon atoms and x=0 or from 1 to 16,

acyl sarcosides having from 8 to 24 carbon atoms in the acyl group,

acyl tauride having from 8 to 24 carbon atoms in the acyl group,

acyl isethionates having from 8 to 24 carbon atoms in the acyl group,

sulfosuccinic acid mono- and/or -dialkyl esters having from 8 to 24 carbon atoms in the alkyl group and sulfosuccinic acid monoalkyl polyoxyethyl esters having from 8 to 24 carbon atoms in the alkyl group and from 1 to 6 oxyethyl groups,

alpha olefin sulfonates having from 8 to 24 carbon atoms,

alkyl sulfates and/or alkyl ether sulfate salts having the formula R—(OCH₂—CH₂)ₙ—O—SO₃X, where R preferably denotes a straight-chained or branched, saturated or unsaturated alkyl group having from 8 to 30 carbon atoms, x denotes the integer 0 or from 1 to 12 and X denotes an alkali-, alkaline earth-, ammonium- or alkanolamine ion,

sulfonates of unsaturated fatty acids with from 8 to 24 carbon atoms and from 1 to 6 double bonds,

esters of tartaric acid and citric acid having alcohols, which are the addition products of from about 2-15 molecules of ethyl oxide and/or propylene oxide on fatty alcohols having from 8 to 22 carbon atoms, and/or

alkyl- and/or alkenyl ether phosphates of the formula

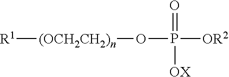

wherein R¹ preferably denotes an aliphatic hydrocarbon radical having from 8 to about 30 carbon atoms, R⁷ for hydrogen, a radical (CH₂CH₂O)ₙR¹ or X, n denotes numbers from 0 to 10 and X denotes hydrogen, an alkali or alkaline earth metal or the group —NR³R⁴R⁵R⁶, with R³ to R⁶ denoting a C₁ to C₄ hydrocarbon radical independently of each other,

Particular preference is given to the mild, high-foam surfactants of the aforementioned group.

With regard to an optimal balance between mild and heavy foam, particular preference is given to alkyl(ether)sulfate surfactants, sulfosuccinate surfactants and/or isethionate surfactants in the aforementioned amounts. Particular preference is given to alkyl(ether)sulfate surfactants.

Therefore, in a second preferred embodiment, cleansing compositions as contemplated herein are exemplified in that the anionic surfactant a) is selected from alkyl(ether)sulfate surfactants, sulfosuccinate surfactants and/or isethionate surfactants, preferably from alkyl(ether)sulfate surfactants.

Particularly suitable alkyl(ether)sulfate surfactants a) have the formula R—(OCH₂—CH₂)ₙ—O—SO₃X, where R preferably denotes a straight-chained or branched, saturated or a simple or multi-unsaturated alkyl alkenyl radical having from 8 to 24 carbon atoms, n denotes 0 or from 1 to 12 and X denotes an alkali-, alkaline earth-, ammonium- or alkanolamine ion.

Particularly preferred alkyl(ether)sulfate surfactants are straight-chained or branched alkylethersulfate salts of the aforementioned formula which contain an alkyl radical R having from 8 to 18 and particularly from 10 to 16 carbon atoms, as well as from 1 to 6 and from 2 to 4 ethylene oxide units, in particular.

Particular preference is given to the sodium, magnesium and/or triethanolamine salts of linear or branched lauryl-, tridecyl- and/or myristylsulfate which have an ethoxylation degree of from 2 to 4, such as the surfactants known by the INCI designation Sodium Laureth Sulfate.

In a further preferred embodiment of the present disclosure, it is particularly advantageous if the cosmetic cleansing compositions contain at least one alkyl(ether)sulfate surfactant of the aforementioned formula in an amount of from about 1.00 to about 10.00 wt. %, preferably from about 3.00 to about 10.00 wt. %, more preferably from about 4.00 to about 10.00 wt. % and particularly from about 5.00 to about 10.00 wt. %, wherein the quantity specifications relate to the total weight of the cosmetic cleansing compositions.

Particularly suitable isethionate surfactants a) correspond to the following formula (II)

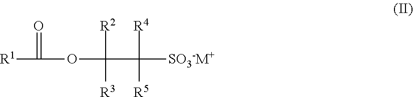

where

the radicals R² to R⁵ denote a hydrogen- or a C₁-C₄-alkyl residue,

R¹ denotes a linear or branched, saturated or unsaturated alkyl radical having from 6 to 30 carbon atoms, and

M³⁰ denotes an ammonium-, alkanol ammonium- or a metal cation.

For particularly mild formulations and particularly good compatibility with other anionic surfactants, particularly with alkyl(ether)sulfate surfactants, it can be advantageous if, in formula (II) at least one of the radicals R² to R⁵ is a C₁-C₄-alkyl radical and the other radicals are independently a hydrogen atom or a C₁-C₄-alkyl radical.

Preferred anionic surfactants of the aforementioned formula (II) have a linear or branched, saturated or unsaturated alkyl radical having from 8 to 18 carbon atoms as radical R¹. It is particularly preferred that the radical R¹ denotes a C₈-, C₁₀-, C₁₂-, C₁₄-, C₁₆-radical or mixtures of these fatty acid radicals as they are obtained, if the fatty acid(s) is(are) derived from natural oils, such as coconut oil.

In further preferred isethionate surfactants of the aforementioned formula (II), the radicals R² to R⁵ each denote hydrogen or a methyl-, ethyl-, n-propyl-, n-butyl- or 2-butyl group.

It is preferable that the radicals R² to R⁵ denote hydrogen or at least one of the radicals R² to R⁵ denotes a methyl-, ethyl- or a n-propyl group and particularly a methyl group.

In a particularly preferred embodiment, one of the radicals R² to R⁵ denotes a C₁-C₄-alkyl group—particularly a methyl group—and the other radicals each denote a hydrogen atom.

In principle, it is also possible for the isethionate surfactant according to formula (II) to contain an isomer mixture in which both components are contained, for example, as radical R², a C₁-C₄-alkyl group, particularly a methyl group, and as residues R³ to R⁵ each have a hydrogen atom, as well as components which, such as radical R⁵, represent a C₁-C₄-alkyl group, in particular a methyl group, and R² to R⁴ each have a hydrogen atom. M⁺ in the aforementioned formula (II) preferably denotes an alkali metal cation or an ammonium ion. It is particularly preferred that the M⁺ denotes a potassium- or a sodium ion and, particularly, a sodium ion.

Particularly referred isethionate surfactants according to the aforementioned formula (II) are the compounds known by the INCI designations Sodium Cocoyl Isethionate, Sodium Lauroyl Isethionate, Sodium Myristyl Isethionate, Sodium Palmitoyl Isethionate, Sodium Stearyl Isethionate, Sodium Oleyl Isethionate, Sodium Cocoyl Methyl Isethionate, Sodium Lauroyl Methyl Isethionate, Sodium Myristyl Methyl Isethionate, Sodium Palmitoyl Methyl Isethionate, Sodium Stearyl Methyl Isethionate and/or Sodium Oleyl Methyl Isethionate.

Particular preference is given to Sodium Cocoyl Methyl Isethionate and/or Sodium Lauroyl Methyl Isethionate.

Corresponding commercial products are available, for example, from the company Innospec under the trade name “Iselux®” and from Clariant or Uniquema under the trade names “Hostapon®” or “Arlatone”.

The isethionate surfactant(s) according to formula (II) can be preferably used in the cosmetic cleansing compositions as contemplated herein in amounts of from about 0.10 to about 5.00 wt. %, more preferably from about 0.25 to about 4.75 wt. %, even more preferably from about 0.50 to about 4.50 wt. % and particularly from about 0.75 to about 4.25 wt. %, wherein the quantity specifications relate to the total weight of the cosmetic cleansing compositions.

Particular preference is given to use of the isethionate surfactants according to formula (II) together with the aforementioned alkyl(ether)sulfate surfactants in the cosmetic cleansing compositions as contemplated herein. In this case, it is advantageous if the alkyl(ether)sulfate surfactants are used in excess for the isethionate surfactants.

Particularly suitable sulfosuccinate surfactants a) correspond to the following formula (III)

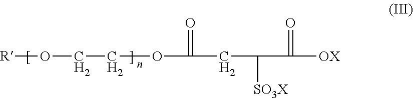

where

R1 denotes a linear or branched, saturated or unsaturated alkyl radical having from 6 to 30 carbon atoms,

n denotes a number from 0 to 20, preferably from 1 to 10, and

X denotes an ammonium-, alkanol ammonium- or a metal cation.

Preferred sulfosuccinate surfactants of the aforementioned formula (II) have a linear or branched, saturated or unsaturated alkyl radical having from 8 to 18 carbon atoms as radical R1. It is particularly preferred that the radical R′ denotes a C₈-, C₁₀-, C₁₂-, C₁₄-, C₁₆-radical or mixtures of these fatty acid radicals as they are obtained, if the fatty acid(s) is(are) derived from natural oils, such as coconut oil.

Preferably, the number n denotes 0 or the number 1, 2, 3, 4, 5, 6, 7 or 8.
M⁺ in the aforementioned formula (III) preferably denotes an alkali metal- or an ammonium ion.

It is particularly preferred that the X denotes a potassium- or a sodium ion and, particularly, a sodium ion.

Particularly preferred sulfosuccinate surfactants according to formula (III) are, for example, the compounds known by the INCI designations Disodium Lauryl Sulfosuccinate, Disodium Laureth Sulfosuccinate and Disodium Cocoyl Sulfosuccinate.

Particular preference is given to Disodium Laureth Sulfosuccinate.
Corresponding commercial products are available, for example, from Evonik under the trade name “Rewopol®” or from BASF under the trade name “Texapon SB®”.

The sulfosuccinate surfactant(s) according to formula (III) can be preferably used in the cosmetic cleansing compositions as contemplated herein in amounts of from about 0.10 to about 5.00 wt. %, more preferably from about 0.25 to about 4.75 wt. %, even more preferably from about 0.50 to about 4.50 wt. % and particularly from about 0.75 to about 4.25 wt. %, wherein the quantity specifications relate to the total weight of the cosmetic cleansing compositions.

Particular preference is given to use of the sulfosuccinate surfactants according to formula (III) together with the aforementioned alkyl(ether)sulfate surfactants in the cosmetic cleansing compositions as contemplated herein. In this case, it is advantageous if the alkyl(ether)sulfate surfactants are used in excess for the sulfosuccinate surfactants.

The cosmetic cleansing compositions as contemplated herein must contain at least one nonionic surfactant b) according to formula (I)

R¹CO—N(R²)—CH₂—CH(OH)—CH(OH)—CH(OH)—CH(OH)—CH₂OH (I),

where

R¹CO denotes a straight-chained or branched, saturated or unsaturated C₈-C₂₂-acyl radical and

R² denotes a C₁-C₄-alkyl radical.

Addition of nonionic surfactants b) to anionic surfactants containing cleansing compositions achieves a significant improvement of haptic and optical properties of the resulting foam. The amount of foam produced by the anionic surfactants is not or only insignificantly reduced by the nonionic surfactants b), but at the same time the foam becomes fine-pored and has less tendency to drainage.

Preferred nonionic surfactants b) according to formula (I) have a C₈-C₁₈-acyl radical as radical R¹, preferably a C₈-C₁₈-acyl radical.

Furthermore, preferred nonionic surfactants b) of formula (I) have a methyl- or ethyl radical as radical R², preferably a methyl radical.
Particularly preferred nonionic surfactants b) according to formula (I) can be selected from the following compounds:

N—C₈-acyl-N-methyl-glucamine, N—C_{8/10}-acyl-N-methyl-glucamine, N—C₁₀-acyl-N-methyl-glucamine, N—C₁₂-acyl-N-methyl-glucamine (INCI designation: Lauroyl Methyl Glucamide), N—C_{12/14}-acyl-N-methyl-glucamine, N—C₁₄-acyl-N-methyl-glucamine (INCI-Myristoyl Methyl Glucamide), N-coco-acyl-N-methyl-glucamine (INCI designation: Cocoyl Methyl Glucamide), N—C₁₆-acyl-N-methyl-glucamine, N—C₁₈-acyl-N-methyl-glucamine (Stearyl Methyl Glucamide or Oleyl Methyl Glucamide), N—C_{12/18}-acyl-N-methyl-glucamine and/or N—C_{16/18}-acyl-N-methyl-glucamine.

Compounds according to formula (I) are commercially available from multiple providers, such as the company Clariant under the trade names Glucamide® or Glucotain®.

In a third preferred embodiment, cosmetic cleansing compositions as contemplated herein are exemplified in that the nonionic surfactant b) is selected from compounds of the aforementioned formula (I), where R¹CO denotes a C₈-C₁₈-acyl radical and R² denotes a methyl radical, preferably from the compounds N—C₈-acyl-N-methyl-glucamine, N—C_{8/10}-acyl-N-methyl-glucamine, N—C₁₀-acyl-N-methyl-glucamine, N—C₁₂-acyl-N-methyl-glucamine, N—C_{12/14}-acyl-N-methyl-glucamine, N—C₁₄-acyl-N-methyl-glucamine, N-Coco-acyl-N-methyl-glucamine, N—C₁₆-acyl-N-methyl-glucamine, N—C₁₈-acyl-N-methyl-glucamine, N—C_{12/18}-acyl-N-methyl-glucamine and/or N—C_{16/18}-acyl-N-methyl-glucamine.

A maximum surfactant content of about 18 wt. %, relative to the total weight of the cosmetic cleansing composition has been found to be a preferred total surfactant content in the cosmetic cleansing compositions as contemplated herein.

In a further preferred embodiment of the present disclosure, therefore, the cosmetic cleansing compositions have a maximum total surfactant content of about 18 wt. %, more preferably a maximum of about 16 wt. %, even more preferably a maximum of about 14 wt. % and particularly a maximum of about 12.5 wt. %, wherein the quantity specifications relate to the total weight of the cosmetic cleansing composition.

An optimum equilibrium between good cleansing and high foam quantities, mildness and fine porosity of the foam of the cleansing agents as contemplated herein could also be achieved, when the at least one anionic surfactant a) is added to the at least one nonionic surfactant b) according to formula (I) in a weight ratio of from about 4.5:1 to about 1:2.

In a fourth preferred embodiment, the weight ratio of the at least one anionic surfactant a) to an at least one nonionic surfactant b) according to formula (I) in the cosmetic cleansing compositions as contemplated herein, therefore, is from about 4.5:1 to about 1:2, preferably from about 4:1 to about 1:1 and particularly from about 3.5:1 to about 1.5:1.

Therefore, particularly preferred cosmetic cleansing compositions are exemplified in that they (relative to their total weight) contain, as surfactants,

a) from about 1.00 to about 10.00 wt. % of at least one alkyl(ether)sulfate and
b) from about 1.00 to about 5.00 wt. % of at least one nonionic surfactant b) according to formula (I), preferably N—C₈-acyl-N-methyl-glucamine, N—C_{8/10}-acyl-N-methyl-glucamine, N—C₁₀-acyl-N-methyl-glucamine, N—C₁₂-acyl-N-methyl-glucamine, N—C_{12/14}-acyl-N-methyl-glucamine, N—C₁₄-acyl-N-methyl-glucamine, N-Coco-acyl-N-methyl-glucamine, N—C₁₆-acyl-N-methyl-glucamine, N—C₁₈-acyl-N-methyl-glucamine, N—C_{12/18}-acyl-N-methyl-glucamine and/or N—C_{16/18}-acyl-N-methyl-glucamine.

The cosmetic cleansing compositions as contemplated herein must contain polyethylene glycol(s) (PEGs) having a high molar mass.

It has been found that PEGs having a high molecular weight derived from the surfactants a) and b) further stabilize the amount of foam formed and further improve the fine porosity of the foam. The foam becomes particularly cream with the addition of PEGs having a high molecular weight and can be easily distributed on the skin. The composition is already distinguished by a pronounced smoothness of during use which supports the nourishing character of the formulation.

The cleaned skin feels particularly smooth, soft and supple after the foam has been rinsed off and the natural skin moisture is kept in balance.

A further advantage is that the combination of the active substances a) to c) in the cleansing compositions as contemplated herein, can produce high-transparency, high-foaming products which are optically particularly appealing.

In conventional mild, low-surfactant systems (which usually contain amphoteric or zwitterionic surfactants), the incorporation of PEGs having a high molecular weight usually results in undesired, unstable, opaque to milky, cloudy products.

“Having a high molecular weight” and thus particularly suitable PEGs for the cosmetic cleansing compositions as contemplated herein is understood to mean polyethylene glycols c) having an average molar mass M_{w} of from about 1,000,000 to about 10,000,000 Dalton. The average molecular weight M_{w} can, for example, be determined by gel permeation chromatography (GPC) with polystyrene as an internal standard according to DIN 55672-3.

Polyethylene glycols c) having an average molar mass M_{w} of from about 1,500,000 to about 8,000,000 Dalton, preferably from about 2,000,000 to about 6,000,000 Dalton and particularly from about 2,500,000 to about 5,000,000 Dalton are suitable.

Particularly suitable polyethylene glycols c) are commercially available, for example, under the trade name Polyox®.

A particularly suitable polyethylene glycol c) is the PEG known by the INCI designation PEG-90M and available, for example, under the trade name Polyox® WSR 301 from the company Dow.

In a fifth preferred embodiment, therefore, cosmetic cleansing compositions as contemplated herein are exemplified in that they contain at least one polyethylene glycol c) having an average molar mass M_{w} of from about 1,500,000 to about 8,000,000 Dalton, preferably from about 2,000,000 to about 6,000,000 Dalton and particularly from about 2,500,000 to about 5,000,000 Dalton.

The compositions as contemplated herein can be produced by simply mixing the essential components for the present disclosure a) to c) with a cosmetic carrier and do not require any additional steps, such as heating.

Furthermore, it is desirable that the viscosity of the cleansing compositions as contemplated herein can be adjusted simply and only by minor variations to different applications, so that the compositions can be adapted to the respective intended use quickly and without difficulty. This can be necessary, for example, when simple removability of the composition is not always assured in equal measured due to the respective type of packaging.

It has been found that the compositions as contemplated herein can be thickened particularly well and variably and maintain a stable viscosity without a negative influence on their outstanding foam properties and excellent distributability on the skin.

Adjustment of the viscosity of the compositions as contemplated herein are particularly successful if associative thickening agents are used instead of salts (such as the compositions normally contained in surfactants) and/or polymers (natural polymers such as xanthan or carrageen or acrylic-based synthetic polymers).

In a sixth preferred embodiment, agents as contemplated herein are exemplified in that they contain (relative to their total weight) an additional from about 0.10 to about 5.00 wt. %, preferably from about 0.20 to about 4.00 wt. % and particularly from about 0.50 to about 3.00 wt. % of at least one associative thickening agent.

Suitable associative thickening agents as contemplated herein are understood to mean

(i) alkoxylated esters of a polyol and at least one C₆-C₃₀-carboxylic acid, wherein the alkoxylated esters have at least one structural unit -(AO)ₘ—, where AO denotes an ethylene oxide group (EO) and/or a propylene oxide group (PO) and m denotes integers between 10 and about 300, or
(ii) alkoxylated C₆-C₃O-dicarboxylic acid esters.

Particularly suitable esters (i) contain, as a polyol component, 1,2-propylene glycol, 1,3-propylene glycol, 1,2-butylene glycol, 1,3-butylene glycol, 1,2-pentanediol, 1,3-pentanediol, 1,2-hexanediol, 1,6-hexanediol, glycerol, diglycerol, polyglycerol, polyethylene glycol, mannitol, isomaltol, sorbitol, xylitol, erythritol, pentaerythritol, glucose, mannose, fructose, ribose, galactose, fucose, rhamnose, saccharose, lactose, maltose and/or trehalose. Particular preference is given to 1,2-propylene glycol, 1,2-butylene glycol, 1,2-hexanediol, 1,6-hexanediol, glycerol, diglycerol, polyethylene glycol, mannitol, sorbitol, erythritol, pentaerythritol, glucose and/or methylglucose, and very particular preference is given to 1,2-propylene glycol, glycerol, sorbitol, pentaerythritol, glucose and/or methylglucose.

Furthermore, esters (i) suitable as contemplated herein are preferably alkoxylated esters, which contain at least one straight-chain or branched, saturated or one mono- or polyunsaturated carboxylic acid as C₆-C₃O-carboxylic acid. C₈-C₂₄-carboxylic acids are preferred and caprylic acid, capric acid are particularly preferred, lauric acid, tridecanoic acid, myristic acid, palmitic acid, stearic acid, arachidic acid, behenic acid, undecylenic acid, myristoleic acid, palmitoleic acid, petroselinic acid, oleic acid, gadoleic acid, linoleic acid, linolenic acid, arachidonic acid and/or mixtures of these acids

As contemplated herein, particular preference is given to C₈-C₂₄-carboxylic acid mixtures which are obtained, if the carboxylic acids are extracted from natural fats and/or oils, such as coconut fat.

Particular preference is given to caprylic acid, capric acid, lauric acid, tridecanoic acid, myristic acid, palmitic acid, stearic acid, behenic acid, oleic acid, gadoleic acid, linoleic acid, linolenic acid, arachidonic acid and/or mixtures of these acids, as obtained when they are extracted from coconut fat.

“Esters” (i) used as contemplated herein—depending on the number of hydroxyl groups in the polyol—are mono-, di-, tri- and/or polyesters which can be alkoxylated one or more times.

Examples of particularly suitable ester (i) are the compounds known by the INCI-designations PEG-120 Methylglucose Dioleate, PEG-120 Methylglucose Trioleate, PEG-(40-60) Sorbitan Tetraoleate, PEG-150 Pentaerythril Tetrastearate, PEG/PPG-120/10 Trimethylolpropane Trioleate, PEG-18 Glyceryl Oleate/Cocoate, PEG-55 Propylene Glycol Oleate, PEG-200 Hydrogenated Glyceryl Palmate and/or PEG-55 Propylene Glycol Oleate, which are commercially available from multiple providers.

Particular preference is given to PEG-120 Methylglucose Trioleate, PEG-150 Pentaerythril Tetrastearate and/or PEG-55 Propylene Glycol Oleate.

Particularly preferred suitable dicarboxylic acid esters (ii) are understood to mean PEG- and/or PPG-dicarboxylic acid esters of straight-chained or branched, saturated or unsaturated C₈-C₂₄-carboxylic acids, particularly C₁₂-C₂₀-carboxylic acids.

Examples of such carboxylic acid esters are the compounds known by the INCI designations PEG-150 Distearate and PEG-400 Dioletate.

In a further preferred embodiment, cosmetic cleansing compositions as contemplated herein are exemplified in that they at least one of the compounds known by the INCI designations PEG-120 Methylglucose Dioleate, PEG-120 Methylglucose Trioleate, PEG-(40-60) Sorbitan Tetraoleate, PEG-150 Pentaerythril Tetrastearate, PEG/PPG-120/10 Trimethylolpropane Trioleate, PEG-18 Glyceryl Oleate/Cocoate, PEG-55 Propylene Glycol Oleate, PEG-200 Hydrogenated Glyceryl Palmate, PEG-55 Propylene Glycol Oleate, PEG-150 Distearate and/or PEG-400 Dioletate as an associative thickening agent, preferably PEG-120 Methylglucose Trioleate, PEG-150 Pentaerythril Tetrastearate and/or PEG-55 Propylene Glycol Oleate, in a percentage by weight of from about 0.10 to about 5.00 wt % of the total weight of the cosmetic cleansing composition.

A particularly preferred embodiment of the present disclosure is exemplified in that the cosmetic cleansing compositions contain (relative to their total weight)

a) from about 1.00 to about 10.00 wt. % of at least one alkyl(ether)sulfate,
b) from about 1.00 to about 5.00 wt. % of at least one nonionic surfactant b) according to formula (I), preferably N—C₈-acyl-N-methyl-glucamine, N—C_{8/10}-acyl-N-methyl-glucamine, N—C₁₀-acyl-N-methyl-glucamine, N—C₁₂-acyl-N-methyl-glucamine, N—C_{12/14}-acyl-N-methyl-glucamine, N—C₁₄-acyl-N-methyl-glucamine, N-Coco-acyl-N-methyl-glucamine, N—C₁₆-acyl-N-methyl-glucamine, N—C₁₈-acyl-N-methyl-glucamine, N—C_{12/18}-acyl-N-methyl-glucamine and/or N—C_{16/18}-acyl-N-methyl-glucamine,
c) from about 0.10 to about 1.00 wt. % of at least one polyethylene glycol c) having an average molar mass M_{w} of from about 2,000,000 to about 6,000,000 Dalton, particularly from about 2,500,000 to about 5,000,000 Dalton and
d) from about 0.10 to about 5.00 wt. % of at least one associative thickening agent selected from PEG-120 Methylglucose Dioleate, PEG-120 Methylglucose Trioleate, PEG-(40-60) Sorbitan Tetraoleate, PEG-150 Pentaerythril Tetrastearate, PEG/PPG-120/10 Trimethylolpropane Trioleate, PEG-18 Glyceryl Oleate/Cocoate, PEG-55 Propylene Glycol Oleate, PEG-200 Hydrogenated Glyceryl Palmate, PEG-55 Propylene Glycol Oleate, PEG-150 Distearate and/or PEG-400 Dioletate.

Furthermore, it was found that the particularly pleasant texture of the cosmetic compositions as contemplated herein can be further optimized if their viscosity is adjusted with a thickening agent system including at least one of the aforementioned associated thickening agents and at least one fatty alcohol polyglycol ether.

Therefore, in a seventh preferred embodiment, cosmetic compositions as contemplated herein are exemplified in that they contain a thickening agent system comprising (relative to the total weight of the cosmetic cleansing composition)

from about 0.10 to about 5.00 wt. % of at least one associative thickening agent and

from about 0.10 to about 5.00 wt. % of at least one fatty alcohol polyglycol ester of the general formula (IV),

R—O—(CH₂—CH₂—O)ₙ—H (IV)

wherein R denotes a straight-chained or branched, saturated or unsaturated C₈-C₂₄-alkyl radical and n denotes a number from 1 to 10.

Preferred fatty alcohol polyglycol ethers of the general formula (IV) are 1-4 times ethoxylated and/or propoxylated, straight-chained or branched, saturated or a mono- or polyunsaturated C₈-C₂₄-alcohol, such as octanol, decanol, lauryl alcohol, myristyl alcohol, cetyl alcohol, stearyl alcohol, arachidyl alcohol, behenyl alcohol, oleyl alcohol and/or elaidyl alcohol.

Particular preference is given to the compounds known by the INCI designation Laureth-(1-4), particularly Laureth-2, Trideceth-(1-4), Myreth-(1-4), especially Myreth-2, Steareth-(1-4), especially Steareth-2 and/or Oleth-(1-4), particularly Oleth-2. Particular preference is given to the compounds known by the INCI designations Laureth-2, Laureth-3, Laureth-4, Trideceth-2, Trideceth-3, Trideceth-4, Myreth-2, Myreth-3 and/or Myreth-4, with particular preference being given to Laureth-2.

In a further preferred embodiment, cosmetic cleansing compositions as contemplated herein are exemplified in that they contain at least one of the compounds known by the INCI designations Laureth-2, Laureth-3, Laureth-4, Trideceth-2, Trideceth-3, Trideceth-4, Myreth-2, Myreth-3 and/or Myreth-4, particularly Laureth-2, as a fatty alcohol polyglycol ester having the general formula (IV).

Therefore, a further particularly preferred embodiment of the present disclosure is exemplified in that the cosmetic cleansing compositions (relative to their total weight)

a) from about 1.00 to about 10.00 wt. % of at least one alkyl(ether)sulfate,
b) from about 1.00 to about 5.00 wt. % of at least one nonionic surfactant b) according to formula (I), preferably N—C₈-acyl-N-methyl-glucamine, N—C_{8/10}-acyl-N-methyl-glucamine, N—C₁₀-acyl-N-methyl-glucamine, N—C₁₂-acyl-N-methyl-glucamine, N—C_{12/14}-acyl-N-methyl-glucamine, N—C₁₄-acyl-N-methyl-glucamine, N-Coco-acyl-N-methyl-glucamine, N—C₁₆-acyl-N-methyl-glucamine, N—C₁₈-acyl-N-methyl-glucamine, N—C_{12/18}-acyl-N-methyl-glucamine and/or N—C_{16/18}-acyl-N-methyl-glucamine,
c) from about 0.10 to about 1.00 wt. % of at least one polyethylene glycol c) having an average molar mass M_{w} of from about 2,000,000 to about 6,000,000 Dalton, particularly from about 2,500,000 to about 5,000,000 Dalton,
d) from about 0.10 to about 5.00 wt. % of at least one associative thickening agent selected from PEG-120 Methylglucose Dioleate, PEG-120 Methylglucose Trioleate, PEG-(40-60) Sorbitan Tetraoleate, PEG-150 Pentaerythril Tetrastearate, PEG/PPG-120/10 Trimethylolpropane Trioleate, PEG-18 Glyceryl Oleate/Cocoate, PEG-55 Propylene Glycol Oleate, PEG-200 Hydrogenated Glyceryl Palmate, PEG-55 Propylene Glycol Oleate, PEG-150 Distearate and/or PEG-400, and
e) from about 0.10 to about 5.00 wt. % of at least one of the compounds known by the INCI designations Laureth-2, Laureth-3, Laureth-4, Trideceth-2, Trideceth-3, Trideceth-4, Myreth-2, Myreth-3 and/or Myreth-4.

To improve the skin-nourishing properties of the agents as contemplated herein, particularly for improvement and/or retention of skin moisture and elasticity, it is advantageous if they also contain at least one skin care substance.

Therefore, in a seventh preferred embodiment, agents as contemplated herein are exemplified in that

they additionally contain at least skin care substance selected from

cationic polymers,

vitamins and/or

glycerol.

Examples of suitable cationic polymers are:

quaternated cellulose derivatives, such as those commercially available under the names Celquat® and Polymer JR®,

hydrophobically modified cellulose derivatives, such as the cationic polymers sold under the trade name SoftCat®,

cationic alkylpolyglycosides,

cationized honey, such as the commercial product Honeyquat® 50,

cationic guar derivatives, such as the products sold under the trade names Cosmedia® Guar N-Hance® and Jaguar®, in particular,

polymeric dimethyldiallyl ammonium salts and the copolymers thereof having esters and amides of acrylic acid and methacrylic acid. The products commercially available under the names Merquat® 100 (poly(dimethyldiallylammoniumchloride)) and Merquat® 550 (dimethyldiallylammoniumchloride-acrylamide-copolymer) are examples of such cationic polymers,

copolymers of vinylpyrrolidon with quaternated derivatives of dialkylaminoalkylacrylate and -methacrylate, such as vinylpyrrolidon-dimethylaminoethylmethacrylate-copolymers quaternated with diethylsulfate. Such compounds are commercially available under the names Gafquat® 734 and Gafquat® 755,

vinylpyrrolidon-vinylimidazoliummethochloride-copolymers, such as those offered under the names Luviquat® FC 370, FC 550, FC 905 and HM 552,

quaternated polyvinyl alcohol,

and the polymers known by the designations

Polyquaternium 2, Polyquaternium 17, Polyquaternium 18, Polyquaternium-24, Polyquaternium 27, Polyquaternium-32, Polyquaternium-37, Polyquaternium 74 and Polyquaternium 89.

Preference is given to cationic polysaccharide polymers such as quaternated cellulose polymers, hydrophobically modified cationic cellulose derivatives and/or cationic guar derivatives, as well as polymeric dimethyldiallylammonium salts and copolymers thereof with esters and amides of acrylic acid and methacrylic acid. Particular is given to cationic polymers known by the INCI designations Guar Hydroxypropyltrimonium Chloride, Polyquaternium-10, Polyquaternium-37, Polyquaternium-67 Polyquaternium-72, Polyquaternium-6 and/or Polyquaternium-7, with particular preference being given to Polyquaternium-7, Polyquaternium-10 and/or Guar Hydroxypropyltrimonium Chloride.

The cationic polymer or polymers can be preferably used in the cleansing compositions as contemplated herein in a percentage by weight of from about 0.01 to about 2 wt. % of the total weight of the cosmetic composition. Weight shares of the cationic polymer or polymers are more preferably from about 0.05 to about 1.50 wt. % and particularly from about 0.10 to about 1.00 wt. %.

In a further preferred embodiment, cosmetic cleansing compositions as contemplated herein are exemplified in that they preferably contain from about 0.01 to about 2.00 wt. %, more preferably from about 0.05 to about 1.50 wt. % and particularly from about 0.10 to about 1.00 wt. % of at least one cationic care polymer (relative to their total weight), preferably being at least one of the cationic care polymers known under the INCI designation Polyquaternium-7, Polyquaternium-10 and/or Guar Hydroxypropyltrimonium Chloride.

The suitable “vitamins” preferably include the following vitamins, provitamins and vitamin precursors, as well as derivatives thereof:

1. Vitamin A: The group of substances called Vitamin A include retinol (Vitamin A₁) and 3,4-didehydroretinol (Vitamin A₂). β-carotin is the provitamin of retinal. Vitamin A—components can include Vitamin A—acid and the esters thereof, Vitamin A—aldehyde) and Vitamin A—alcohol, as well as the esters thereof, such as palmitate and acetate.
2. Vitamin B: The Vitamin B Group or Vitamin B complex include

Vitamin B₁ (Thiamine)

Vitamin B₂ (Riboflavin)

Vitamin B₃. The compounds nicotinic acid amide (niacin amide) are often carried under this designation.

Vitamin B₅ (pantothenic acid and panthenol). Among this group, panthenol is preferably used. Usable derivatives of panthenol include, in particular, the esters and ethers of panthenol, pantolacton, as well as cationically derived panthenols. Specific examples are panthenoltriacetate, panthenolmonoethylether and the monoacetate thereof, as well as cationic panthenol derivatives.

Vitamin B₆ (pyridoxine, as well as pyridoxamine and pyridoxal).

Vitamin C (ascorbic acid): use in the form of palmitic acid ester, glucoside or phosphate can be preferred. Use in combination with tocopherols can also be preferred.

Vitamin E (tocopherols, more particularly α-tocopherol).

Vitamin F: The term “Vitamin F” usually means essential fatty acids, more particularly linoleic acid, linolenic acid and arachidon acid.

Vitamin H: The compound (3aS,4S, 6aR)-2-oxohexahydrothienol[3,4-d]-imidazol-4-valeric acid, which has however since taken on the trivial name of biotin, is designated as Vitamin H.

Particular preference is given to vitamins, provitamins and vitamin precursors form the groups A, B, E and H. Particular preference is given to vitamins of the B group, such as nicotinic acid amide and/or panthenol, where nicotinic acid amide especially preferred.

The percentage by weight of the vitamins(s), vitamin derivatives(e), and/or the vitamin precursor(s) of the total weight of the cosmetic cleansing compositions as contemplated herein is preferably from about 0.005 to about 2.00 wt. %, more preferably from about 0.006 to about 1.50 wt. %, even more preferably from about 0.008 to about 1.00 wt. % and particularly from about 0.01 to about 0.50 wt.-%.

Glycerol can be added separately to the compositions as contemplated herein as an active care ingredient or it can be a component of the cosmetic carrier.

The content of glycerol in the cosmetic cleansing compositions as contemplated herein is preferably from about 0.10 to about 10 wt. %, more preferably from about 0.25 to about 7.50 wt. % and particularly from about 0.50 to about 5.00 wt. % (relative to the total weight of the cosmetic cleansing composition).

In a further preferred embodiment, cosmetic cleansing compositions as contemplated herein are exemplified in that they preferably contain—relative to their total weight—from about 0.10 to about 10 wt. %, more preferably from about 0.25 to about 7.50 wt. % and particularly from about 0.50 to about 5.00 wt. % glycerol and/or from about 0.01 to about 1.00 wt. %, more preferably from about 0.01 to about 0.50 wt. % and particularly from about 0.01 to about 0.25 wt. % of at least one vitamin, preferably at least one vitamin of the B group, particularly niacinamide and/or panthenol and especially niacinamide.

Transparent compositions are desired for some application forms. Therefore, a further preferred embodiment of the present disclosure is exemplified in that the cosmetic cleansing compositions are transparent.

The term transparent or translucent in the context of the present disclosure is understood to mean a composition having an NTU value of less than 100. The NTU value (Nephelometric Turbidity Unit) is one of the units used for turbidity measurements in liquids in water treatment. It is the unit of a turbidity of a liquid measured with a nephelometer.

Preferred cosmetic cleansing compositions as contemplated herein are exemplified in that they are transparent or translucent and have an NTU value of less than 100, preferably less than 50.

With the incorporation of PEGs having high molar mass into compositions containing surfactants, the transparency of the compositions can be lost. This effect must be closely monitored when the compositions contain amphoteric and/or zwitterionic surfactants in addition to anionic surfactants.

Therefore, in order to avoid the turbidity effect—particularly in the production of transparent compositions—it is advantageous if the compositions as contemplated herein contain substantially no amphoteric and/or zwitterionic surfactants.

Therefore, an eighth preferred embodiment is exemplified in that amphoteric and/or zwitterionic surfactants, particularly betaine surfactants, are substantially absent from the cosmetic cleansing compositions as contemplated herein.

“Substantially absent” in the context of the present disclosure is understood to mean that the cosmetic cleansing compositions preferably contain (relative to their total weight) less than about 2.00 wt. %, more preferably less than about 1.00 wt. %, even more preferably less than about 0.50 wt. % and, in particular, less than about 0.10 wt. % amphoteric and/or zwitterionic surfactants, particularly betaine surfactants.

To further improve the mildness of the cleansing compositions as contemplated herein, it can be advantageous in a further preferred embodiment if they also contain—relative to their total weight—from about 0.01 to about 5.00 wt. %, preferably from about 0.05 to about 4.00 wt. %, more preferably from about 0.10 to about 3.00 wt. % and particularly from about 0.25 to about 2.50 wt. % of at least one additional nonionic surfactant differing from b).

Examples of suitable additional nonionic surfactants differing from b) include

aminoxides,

fatty acid alkanolamides having the following general formula,

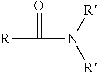

wherein R preferably denotes a linear or branched, saturated or unsaturated alkyl- or alkenyl radical having from 8 to 24 carbon atoms and the radical R′ denotes hydrogen or the group —(CH₂)ₙOH, where n denotes the number 2 or 3, provided that at least one of the radicals R′ denotes the aforementioned radical—(CH₂)ₙOH,

addition products of ethylene oxide on fatty acid alkanolamides and fatty amines, and/or

alkyl(oligo)glucosides,

addition products of from 5 to about 60 mol of ethylene oxide on hardened castor oil,
partial esters of polyols having 3-6 carbon atoms with saturated fatty acids having 8 to 22 carbon atoms,
sterols. Sterols are understood to mean a group of steroids that carries at least one hydroxyl group on carbon atom 3 of the steroid structure and are isolated from both animal tissue (zoosterols) and plant fats (phytosterols). Examples of zoosterols are cholesterol and lanosterol. Examples of suitable phytosterols are ergosterol, stigmasterol and sitosterol. Sterols are also isolated from fungi and yeasts, so-called mykosterols.
Phospholipids. This includes, in particular glucose phospholipids extracted, for example, from egg yolk or plant seeds (e.g. soybeans), such as lecithins or phosphoric cholines.

Suitable alkyl(oligo)glycosides can be preferably selected from compounds of the general formula of RO-[G]ₓ, wherein [G] is preferably derived from aldoses or ketoses having 5 or 6 carbon atoms, preferably glucose.

The index value x denotes the oligomerization degree (DP), i.e. for the distribution of mono and oligoglycosides. The index value x preferably has a value in the range from 1 to 10, more preferably in the range from 1 to 3, wherein said value is not an integer but can be a fraction, which can be determined analytically.

Particularly preferred alkyl(oligo)glycosides have an oligomerization degree of between about 1.2 and about 1.5.

The radical R preferably denotes at least one alkyl- and/or alkylene radical having from 4 to 24 carbon atoms.

Particularly preferred alkyl(oligo)glycosides are the compounds known under the INCI designations Caprylyl/Capryl Glucoside, Decyl Glucoside, Lauryl Glucoside and Coco Glucoside.

Suitable aminoxides can be selected from at least one compound of the general formulae (I) or (II)

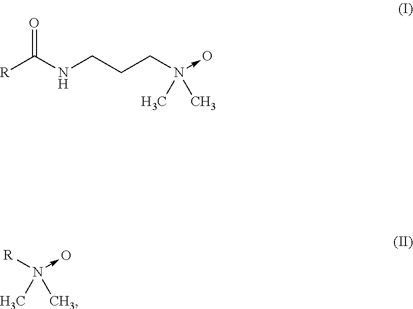

wherein R denotes a straight-chained or branched, saturated or mono- or polyunsaturated alkyl- or alkenyl radical having from 6 to about 24 carbon atoms, preferably from 8 to 18 carbon atoms in each case.

Particular preference is given to the surfactants of the aforementioned formula (I) or (II) known under the INCI designations Cocamine Oxide, Lauramine Oxide and/or Cocamidopropylaminoxide.

Particularly preferred nonionic surfactants which can be contained in the compositions as contemplated herein are alkyl(oligo)glucosides, particularly the compounds known under the INCI designations Caprylyl/Capryl Glucoside, Decyl Glucoside, Lauryl Glucoside and/or Coco Glucoside.

In addition to the aforementioned essential and optional components the cosmetic cleansing compositions as contemplated herein can, in a further preferred embodiment, contain at least one additional conditioning active ingredient, preferably a skin-conditioning active ingredient, from the group of natural, synthetic and/or mineral oil-, fat- and/or wax components to further improve the nourishing properties of the agents.

Suitable natural (plant) oils are normally understood to mean triglycerides and mixtures of triglycerides. Preferred natural oils are coconut oil, (sweet) almond oil, walnut oil, peach kernel oil, apricot kernel oil, avocado oil, tee tree oil, soja oil, sesame oil, sunflower oil, tsubaki oil, evening primrose oil, rice bran oil, palm kernel oil, mango kernel oil, meadowfoam seed oil, thistle oil, macadamia nut oil, grape seed oil, amaranth seed oil, argan oil, bamboo oil, olive oil, wheatgerm oil, pumpkin seed oil, mallow oil, hazelnut oil, safflower oil, canola oil, sasanqua oil, jojoba oil, rambutan oil, cocoa butter and shea butter. Preference is given to almond oil, jojoba oil, coconut oil and/or shea butter.

Mineral oils, paraffin and iso-paraffin oils, as well as synthetic hydrocarbons are preferably used as mineral oils. An example of a usable hydrocarbon is the commercially available 1,3-Di-(2-ethylhexyl)-cyclohexan (Cetiol® S).

A dialkylether can also be used as an oil component.

Usable dialkylethers are particularly di-n-alkylether with a total of between 12 and 36 C-atoms, more particularly from 12 to 24 C-atoms, such as for example di-n-octylether, di-n-decylether, di-n-nonylether, di-n-undecylether, di-n-dodecylether, n-hexyl-n-octylether, n-octyl-n-decylether, n-decyl-n-undecylether, n-undecyl-n-dodecylether and n-hexyl-n-undecylether, as well as di-tert.-butylether, di-iso-pentylether, di-3-ethyldecylether, tert.-butyl-n-octylether, iso-pentyl-n-octylether and 2-methylpentyl-n-octylether.

The commercially available di-n-octylether under the trade name Cetiol® OE is particularly preferred.

Fatty substances are fatty acids, fatty alcohols, as well as natural and synthetic waxes, which can exist both in solid form and liquid form on hydrous dispersions. Linear and/or branched, saturated and/or unsaturated fatty acids with from 6-30 carbon atoms can be used as fatty acids. Fatty acids with from 10-22 carbon atoms are preferred. These include the isostearic acids, such as the commercially available Emersol® 871 and Emersol® 875, and isopalmitic acids, such as the commercially available Edenor® IP 95, as well as all other fatty acids sold under the trade name Edenor® (Cognis). Other typical examples of such fatty acids are caproic acid, caprylic acid, 2-ethyl hexane acid, capric acid, lauric acid, isotridecaric acid, myristiric acid, palmitic acid, palmitoleic acid, stearic acid, isostearic acid, oleic acid, elaidic acid, petroselic acid, linoleic acid, linolenic acid, elaeostearic acid, arachine acid, gadoleic acid, behenic acid and erucic acid, as well as the technical mixtures thereof. The fatty acid fractions that can be obtained from coconut oil or palm oil are usually preferred; the use of stearic acid is usually more preferred.

Saturated, mono- or poly-unsaturated, branched or unbranched fatty alcohols with C₆-C₃₀—, preferably C₁₀-C₂₂ and most preferably C₁₂-C₂₂ carbon atoms, can be used as fatty alcohols. Decanol, octanol, octenol, dodecenol, decenol, octadienol, dodecadienol, decadienol, oleylalcohol, erucaalcohol, ricinolalcohol, stearylalcohol, isostearylalcohol, cetylalcohol, laurylalcohol, myristylalcohol, arachidylalcohol, caprylalcohol, caprinalcohol, linoleylalcohol, linolenylalcohol and behenylalcohol, as well as the guerbetalcohols thereof, can be used. This list contains purely examples and is not exhaustive. The fatty alcohols originate, however, from natural fatty acids, wherein the recovery from the esters of fatty acids by means of reduction can normally be assumed. As contemplated herein, fatty alcohol fractions produced by reducing naturally-occurring triglycerides such as beef tallow, palm oil, peanut oil, rapeseed oil, cotton seed oil, soya oil, sunflower oil and linseed oil, or the fatty acid esters produced from the trans-esterification products with corresponding alcohols can also be used. These substances therefore constitute a mixture of different fatty alcohols. Such substances include those commercially available under the trade names of Stenol®, e.g. Stenol® 1618 or Lanette®, e.g. Lanette® 0 or Lorol®, e.g. Lorol® C8, Lorol® C14, Lorol® C18, Lorol® C8-18, HD-Ocenol®, Crodacol®, z.B. Crodacol® CS, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16 oder Isocarb® 24. As contemplated herein, wool wax alcohols, such as those commercially available under the trade names of Corona®, White Swan®, Coronet® or Fluilan®, can of course also be used.

Solid paraffins or iso-paraffins, carnauba waxes, beeswaxes, candelilla waxes, ozokerites, ceresine, spermaceti, sunflower wax, fruit wax, such as apple wax or citrus wax, micro-waxes from PE- or PP can also be used as natural or synthetic waxes. Such waxes are available from Kahl & Co., Trittau, for example.

### Other Solids Include

ester oils. Ester oils include the esters from C₆-C₃₀ fatty acids with C₂-C₃₀-fatty alcohols. The mono esters of fatty acids with alcohols with 2 to 24 C-atoms are preferred. Examples of used fatty acid fractions in the esters are caproic acid, caprylic acid, 2-ethyl hexane acid, capric acid, lauric acid, isotridecaric acid, myristiric acid, palmitic acid, palmitoleic acid, stearic acid, isostearic acid, oleic acid, elaidic acid, petroselic acid, linolic acid, linolenic acid, elaeostearic acid, arachine acid, gadoleic acid, behenic acid and erucic acid, as well as the technical mixtures thereof.

Examples of the fatty alcohol fractions in the ester oils are isopropylalcohol, capronalcohol, aprylalcohol, 2-ethylhexylalcohol, caprinalcohol, laurylalcohol, isotridecylalcohol, myristylalcohol, cetylalkohol, palmoleylalcohol, stearylalcohol, isostearylalcohol, oleylalcohol, elaidylalcohol, petroselinylalcohol, linolylalcohol, linolenylalcohol, elaeostearylalcohol, arachylalcohol, gadoleylalcohol, behenylalcohol, erucylalcohol and brassidylalcohol, as well as technical mixtures thereof. Isopropylmyristate (Rilanit® IPM), Isononan acid-C16-18-alkylester (Cetiol® SN), 2-ethylhexylpalmitate (Cegesoft® 24), stearic acid-2-ethylhexylester (Cetiol® 868), eetyloleate, glyceroltricaprylate, coconut fatty alcohol-caprinate/-caprylate (Cetiol® LC), n-butylstearate, oleylerucate (Cetiol® J 600), isopropylpalmitate (Rilanit® IPP), Oleyl Oleate (Cetiol®), lauric acid hexylester (Cetiol® A), Di-n-butyladipate (Cetiol® B), myristylmyristate (Cetiol® MM), cetearyl isononanoate (Cetiol® SN), oleic acid decylester (Cetiol® V).

Dicarboxylic acid esters such as di-n-butyladipate, ei-(2-ethylhexyl)-adipate, di-(2-ethylhexyl)-succinate and Di-isotridecylacelaate, as well as diolesters such as ethylenglykol-dioleate, ethylenglykol-di-isotridecanoate, propylenglykol-di(2-ethylhexanoate), propylenglykol-di-isostearate, propylenglykol-di-pelargonate, butanediol-di-isostearate, neopentylglykoldicaprylate,

symmetrical, asymmetrical or cyclical esters of carboxylic acids with fatty alcohols,

glycerolcarbonate or dicaprylylcarbonate (Cetiol® CC),

ethoxylated or non-ethoxylated mono- di- and tri-fatty acid esters of saturated and/or unsaturated linear and/or branched fatty acids with glycerol, such as Monomuls® 90-018, Monomuls® 90-L12, Cetiol® HE or Cutina® MD.

Suitable synthetic oils are preferably silicones, such as

(i) polyalkylsiloxanes, polyarylsiloxanes, polyalkylarylsiloxanes, which are volatile, non-volatile, straight-chained, branched or cyclical, cross-linked or not cross-linked;
(ii) polysiloxanes, the general structure of which contains one or more organofunctional groups, which can be selected from:
a) substituted or unsubstituted aminated groups;
b) (Per)fluorinated groups;
c) thiol groups;
d) carboxylate groups;
e) hydroxylated groups;
alkoxylated groups;
g) acyloxyalkyl groups;
h) amphoteric groups;
i) bisulfite groups;
j) hydroxyacylamino groups;
k) carboxy groups;
l) sulfonic acid groups; and
m) sulfate or thiosulfate groups;
(iii) linear polysiloxane(A)-polyoxyalkylene(B)-block copolymers of type (A-B)ₙ where n>3;
(iv) grafted silicone polymers with a non-silicone-containing, organic backbone including an organic main chain, which is formed from organic monomers, which contain no silicone, to which at least one polysiloxane macromer has been grafted within the chain and also, where applicable, to at least one chain end;
(v) grafted silicone polymers with polysiloxane backbone, to which non-silicone-containing, organic monomers have been grafted, which have a polysiloxane main chain, to which at least one organic macromer, which contains no silicone, has been grafted within the chain and also, where applicable, to at least one chain end;
(vi) or mixtures thereof.

The natural, synthetic and/or mineral oil-, fat- and/or wax components can be preferably used in the cosmetic cleansing compositions as contemplated herein in an amount of from about 0.005 to about 10.00 wt. %, more preferably from about 0.0075 to about 7.50 wt. % and particularly from about 0.01 to about 5.00 wt. %, relative to their total weight.

The cosmetic cleansing agents as contemplated herein preferably contain at least one natural oil.

The cosmetic cleansing agents as contemplated herein preferably have a pH value close to neutral or in the slightly acidic range in order to have as little influence as possible on the balance in the skin flora.

Therefore, a further preferred embodiment is exemplified in that the cosmetic cleansing compositions as contemplated herein have a pH value in the range of from about 4.0 to about 8.0, more preferably from about 4.2 to about 7.0, even more preferably from about 4.3 to about 6.0 and particularly from about 4.5 to about 5.0.

The preferred viscosity range of the cosmetic cleansing compositions as contemplated herein is from about 5,000-13,000 mPas (measures at 20° C. with a Haake Viscotester 550 with a shear stress of 8 s⁻¹). As contemplated herein, cosmetic cleansing compositions in this viscosity range does not draw any threads and do not have any undesired pronounced gel structure.

Other active ingredients, adjuvants and additives, which can be preferably contained in the cleansing compositions as contemplated herein, include:

UV filters,

perfumes,

preservatives such as benzoic acids and/or salialic acids and salts thereof

structurants such as malic acid and lactic acid,

dyes for coloring the agent,

substances for setting the pH value, for example α- and β-hydroxycarboxylic acids, such as citric acid, lactic acid, malic acid, glycolic acid,

active ingredients such as bisabolol and/or allantoin,

complexing agents such as EDTA, NTA, β-alaninediacetic acid and phosphonic acids,

ceramides. Ceramides include N-acylsphingosine (fatty acid amides of sphingosine) or synthetic analogs of such lipids (so-called pseudo-ceramides),

propellants such as propane-butane mixtures, N₂O, dimethylether, CO₂ and air,

antioxidants,

plant extracts,

pearlescent agents such as EGDS or PEG-3 distearate,

opacifiers such as the compounds known under the INCI designation Styrene/Acrylates Copolymer,

viscosity regulators such as electrolytic salts (NaCl).

### Examples

The following cosmetic cleansing compositions as contemplated herein were produces (quantity specifications relative to percentage by weight unless otherwise indicated).

| | 1. | 2 | 3 | 4 | 5 | 6 | 7 | 8 |
|---|---|---|---|---|---|---|---|---|
| Sodium | 7.00 | 7.40 | 9.10 | 9.10 | 5.60 | 7.00 | 9.10 | 7.00 |
| Laureth | | | | | | | | |
| Sulfate (2 EO) | | | | | | | | |
| Lauroyl | 2.50 | 2.50 | 3.00 | 3.00 | 3.70 | 3.00 | | 2.50 |
| Methyl | | | | | | | | |
| Glucamide | | | | | | | | |
| Oleyl Methyl | | | | | | | 4.00 | |
| Glucamide | | | | | | | | |
| PEG-90M | 0.10 | 0.10 | 0.10 | 0.10 | 0.10 | 0.10 | 0.10 | 0.05 |
| Cocamide | 1.50 | 1.70 | | | | | | 1.50 |
| MEA | | | | | | | | |
| Glycerol | 1.00 | 1.00 | 1.00 | 1.00 | 1.00 | 1.00 | 1.00 | 1.00 |
| PEG-120 | 0.50 | 0.25 | | | 1.00 | 1.00 | 2.50 | 0.50 |
| Methyl | | | | | | | | |
| Glucose | | | | | | | | |
| Dioleate | | | | | | | | |
| PEG-200 | | | | 1.70 | | | | |
| Hydrogenated | | | | | | | | |
| Glyceryl | | | | | | | | |
| Palmate | | | | | | | | |
| Laureth-2 | | | 2.50 | | 0.50 | 1.00 | | |
| PEG-7 | | | | 0.40 | | | | |
| Glyceryl | | | | | | | | |
| Cocoate | | | | | | | | |
| Citric Acid | 0.35 | 0.35 | 0.36 | 0.36 | 0.25 | 0.25 | 0.36 | 0.35 |
| Sodium | 0.35 | 0.35 | 0.20 | 0.20 | 0.20 | 0.20 | 0.20 | 0.35 |
| Benzoate | | | | | | | | |
| Sodium | 0.20 | 0.20 | 0.20 | 0.20 | 0.20 | 0.20 | 0.20 | 0.20 |
| Salicylate | | | | | | | | |
| Niacinamide | 0.01 | 0.01 | 0.01 | 0.01 | 0.01 | 0.01 | 0.01 | 0.01 |
| Panthenol | | | | | | 0.20 | | |
| Poly- | | | | | | | | 0.05 |
| quaternium-7 | | | | | | | | |
| Almond oil | | | | | | | | 0.01 |
| Glycol | | 0.45 | | | | | | |
| Distearate | | | | | | | | |
| Styrene/ | | 0.40 | | | | | | |
| Acrylates | | | | | | | | |
| Copolymer | | | | | | | | |
| Perfume | q.s. | q.s. | q.s. | q.s. | q.s. | q.s. | q.s. | q.s. |
| Water | ad | ad | ad | ad | ad | ad | ad | ad |
| | 100 | 100 | 100 | 100 | 100 | 100 | 100 | 100 |

Compositions 1 to 8 as contemplated herein are exemplified by especially good foam properties, a pronounced smooth feeling of the skin during and after application of the agents and very good rinsability. After use, they leave a nourished skin feeling without drying out the skin.

The pH value of the compositions is 5.0±0.3 and the viscosity can be adjusted in a range between 5,000 and 13,000 mPas (measured at 20° C. on a Haake Viscotester 550 shear stress 8s⁻¹).

All formulations are clear/transparent before an optional addition of Styrene/Acrylates Copolymer or Glycol Distearate.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. Cosmetic cleansing composition, comprising
a) at least one anionic surfactant,
b) at least one nonionic surfactant having the following formula (I)
R¹CO—N(R²)—CH₂—CH(OH)—CH(OH)—CH(OH)—CH(OH)—CH₂OH (I),
where
R¹CO denotes a straight-chained or branched, saturated or unsaturated C₈-C₂₂-acyl radical and
R² denotes a C₁-C₄-alkyl radical and
c) at least on polyethylene glycol of a molar mass M_{W} with an average mass of from about 1,000,000 to about 10,000,000 Dalton.

2. Cosmetic cleansing composition according to claim 1, wherein the composition comprises relative to its total weight
from about 1.00 to about 15.00 wt. % of the at least one anionic surfactant a),
from about 0.50 to about 7.50 wt. % of the at least one nonionic surfactant (b) of the aforementioned formula (I), and
from about 0.01 to about 1.00 wt. % of the at least one polyethylene glycol (c) of a molar mass M_{W} with an average mass of 1,000,000 to 10,000,000 Dalton.

3. Cosmetic cleansing composition according to claim 1, wherein the anionic surfactant a) is selected from alkyl(ether)sulfate surfactants, sulfosuccinate surfactants and/or isethionate surfactants.

4. Cosmetic cleansing composition according to claim 1, wherein the nonionic surfactant b) is selected from compounds of the aforementioned formula (I), where R¹CO denotes a C₈-C₁₈-acyl radical and R² denotes a methyl radical, preferably from the compounds N—C₈-acyl-N-methyl-glucamine, N—C_{8/10}-acyl-N-methyl-glucamine, N—C₁₀-acyl-N-methyl-glucamine, N—C₁₂-acyl-N-methyl-glucamine, N—C_{12/14}-acyl-N-methyl-glucamine, N—C₁₄-acyl-N-methyl-glucamine, N-Coco-acyl-N-methyl-glucamine, N—C₁₆-acyl-N-methyl-glucamine, N—C₁₈-acyl-N-methyl-glucamine, N—C_{12/18}-acyl-N-methyl-glucamine and/or N—C_{16/18}-acyl-N-methyl-glucamine.

5. Cosmetic cleansing composition according to claim 1, wherein the composition comprises the at least one anionic surfactant a) and the at least one nonionic surfactant b) according to formula (I) in a weight ratio of from about 4.5:1 to about 1:2.

6. Cosmetic cleansing composition according to claim 1, wherein the composition comprises the at least one polyethylene glycol c) having an average molar mass M_{w} of from about 2,500,000 to about 5,000,000 Dalton.

7. Cosmetic cleansing composition according to claim 1, wherein the composition additionally comprises—relative to its total weight—from about 0.10 to about 5.00 wt. % of at least one associative thickening agent.

8. Cosmetic cleansing composition according to claim 7 wherein the composition comprises at least one of the compounds known by the INCI designations PEG-120 Methylglucose Dioleate, PEG-120 Methylglucose Trioleate, PEG-(40-60) Sorbitan Tetraoleate, PEG-150 Pentaerythril Tetrastearate, PEG/PPG-120/10 Trimethylolpropane Trioleate, PEG-18 Glyceryl Oleate/Cocoate, PEG-55 Propylene Glycol Oleate, PEG-200 Hydrogenated Glyceryl Palmate, PEG-55 Propylene Glycol Oleate, PEG-150 Distearate and/or PEG-400 Dioletate as the at least one associative thickening agent, in a percentage by weight of from about 0.10 to about 5.00 wt % of the total weight of the cosmetic cleansing composition.

9. Cosmetic cleansing composition according to claim 1, wherein the composition comprises a thickening agent system, which, relative to the total weight of the cosmetic cleansing composition, comprises
from about 0.10 to about 5.00 wt. % of at least one associative thickening agent and
from about 0.10 to about 5.00 wt. % of at least one fatty alcohol polyglycol ester of the general formula (IV),
R—O—(CH₂—CH₂—O)ₙ—H (IV)
wherein R denotes a straight-chained or branched, saturated or unsaturated C₈-C₂₄-alkyl radical and n denotes a number from 1 to 10.

10. Cosmetic cleansing composition according to claim 9, wherein the composition comprises at least one of the compounds known by the INCI designations Laureth-2, Laureth-3, Laureth-4, Trideceth-2, Trideceth-3, Trideceth-4, Myreth-2, Myreth-3 and/or Myreth 4 as the at least one fatty alcohol polyglycol ester having the general formula (IV).

11. Cosmetic cleansing composition according to claim 1, wherein the composition additionally comprises at least one skin care substance, selected from
cationic polymers,
vitamins and/or
glycerol.

12. Cosmetic cleansing composition according to claim 11 wherein the composition comprises—relative to its total weight—from about 0.01 to about 2.00 wt. % of at least one cationic care polymer.

13. Cosmetic cleansing composition according to claim 11 wherein the composition comprises—relative to its total weight—from about 0.10 to about 10 wt. % of at least one vitamin.

14. Cosmetic cleansing composition according to claim 1, wherein the composition is transparent.

15. Cosmetic cleansing composition according to claim 1, wherein amphoteric and/or zwitterionic surfactants are substantially absent from the cosmetic cleansing composition.

16. Cosmetic cleansing composition according to claim 1, wherein the composition comprises—relative to its total weight—
from about 5.00 to about 10.00 wt. % of the at least one anionic surfactant a),
from about 1.00 to about 5.00 wt. % of the at least one nonionic surfactant (b) of the aforementioned formula (I), and
from about 0.05 to about 0.30 wt. % of the at least one polyethylene glycol (c) of the molar mass M_{W} with the average mass of from about 1,000,000 to about 10,000,000 Dalton.

17. Cosmetic cleansing composition according to claim 1, wherein the anionic surfactant a) is an alkyl(ether)sulfate surfactant.

18. Cosmetic cleansing composition according to claim 1, wherein the composition additionally comprises—relative to its total weight—from about 0.50 to about 3.00 wt. % of at least one associative thickening agent.

19. Cosmetic cleansing composition according to claim 7, wherein the composition comprises at least one of the compounds known by the INCI designations PEG-120 Methylglucose Trioleate, PEG-150 Pentaerythril Tetrastearate and/or PEG-55 Propylene Glycol Oleate as the associative thickening agent, in a percentage by weight of from about 0.10 to about 5.00 wt % of the total weight of the cosmetic cleansing composition.

20. Cosmetic cleansing composition, comprising
a) at least one anionic surfactant selected from alkyl(ether)sulfate surfactants, sulfosuccinate surfactants and/or isethionate surfactants,
b) at least one nonionic surfactant having the following formula (I)
R¹CO—N(R²)—CH₂—CH(OH)—CH(OH)—CH(OH)—CH(OH)—CH₂(OH) (I),
where
R¹CO denotes a straight-chained or branched, saturated or unsaturated C₈-C₁₈-acyl radical and
R² denotes a methyl radical,
c) at least on polyethylene glycol of a molar mass M_{W} with an average mass of from about 1,000,000 to about 10,000,000 Dalton,
d) from about 0.10 to about 5.00 wt. % of at least one associative thickening agent, and
optionally, from about 0.10 to about 5.00 wt. % of at least one fatty alcohol polyglycol ester of the general formula (IV),
R—O—(CH₂—CH₂—O)ₙ—H (IV)
wherein R denotes a straight-chained or branched, saturated or unsaturated C₈-C₂₄-alkyl radical and n denotes a number from 1 to 10.
